(19) United States
(12) Patent Application Publication
(10) Pub. No.: US 2009/0198251 A1
(43) Pub. Date: Aug. 6, 2009

(54) LEAD DELIVERY, FIXATION AND EXTRACTION DEVICES AND METHODS FOR USE WITH INTRAVASCULAR IMPLANTABLE MEDICAL DEVICES

(76) Inventors: Terrance Ransbury, Chapel Hill, NC (US); Ryan McCormick, Durham, NC (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/367,475

(22) Filed: Feb. 6, 2009

Related U.S. Application Data
(60) Provisional application No. 61/026,606, filed on Feb. 6, 2008.

(51) Int. Cl. A61B 19/00

(52) U.S. Cl. 606/129

(57) ABSTRACT

A lead delivery and fixation device for use with an intravascular implantable device includes a sidecar having a selectively retractable fixation element. A lead is releasably coupled to the sidecar, with an electrode portion exposed for delivering a stimulation therapy. A manipulable catheter is coupled to the fixation element and configured to advance and withdraw a helix portion of the fixation element. The catheter and fixation element are offset from and generally parallel to the lead. The lead is separable from the sidecar in the event that extraction is required.

### RELATED APPLICATIONS

The present invention claims priority to U.S. Provisional Patent Application No. 61/026,606, entitled “Lead Delivery, Fixation and Extraction Devices and Methods for Use ith Intravascular Implantable Medical Devices,” filed Feb. 6, 2008, which is hereby incorporated by reference.

### FIELD OF THE INVENTION

The present invention relates generally to implantable leads for electrical stimulation. More specifically, the present invention relates to methods and devices for delivery, fixation and extraction of cardiac leads for use with intravascular implantable medical devices.

### BACKGROUND OF THE INVENTION

Implantable cardiac rhythm management (CRM) devices such as artificial pacemakers and implantable cardioverter-defibrillators (ICD's) rely on leads for sensing and/or delivering therapy. Conventional pacemakers and ICDs are implanted subcutaneously, typically in the pectoral region. Conventional implantable pulse generators such as pacemakers and ICDs use conventional leads in the form of elongated, floppy lead bodies that insulate, seal and protect one or more conductors which transmit electrical pulses between the pulse generator and one or more electrodes on the lead. The one or more intravascular leads associated with a conventional pacemaker device or ICD are typically not integrated with the device; instead, a header is provided on the device for connecting the one or more leads to the device. The lead tip is affixed in, on, or near the heart, depending on the desired treatment.

Implantation of the one or more intravascular leads for a conventional CRM device involves delivery of the lead to a desired location, followed by fixation of the lead. For a CRM device implanted subcutaneously in the pectoral region, the most common path for delivering the lead into the heart begins at a transvenous incision into the subclavian vein, through the superior vena cava, and down into the right atrium of the heart. Most intravascular cardiac leads for conventional CRM devices are guided through the vasculature with use of a stylet that is inserted into a lumen within the lead body accessed via the proximal end of the lead, with the stylet used to direct the distal end of the lead into the desired position.

Once in position, the distal end of the lead is fixed in position within, on or near the heart, by either passive fixation or active fixation. Passive fixation leads may feature protruding tines and/or hooks on the distal end, such that when the lead tip is inserted to the desired location, biological processes in the heart tissue will secure the lead in place. Active fixation leads typically include a helix or corkscrew tip, and this tip is secured directly into the myocardium. Active fixation offers more precise placement of the lead, as well as greater stability when secured in the heart. Early active fixation leads were secured by rotation of the lead body to engage the corkscrew in the heart. Current active fixation leads for CRM devices typically includes a deployable fixation element, actuated via the stylet from within the central lumen. Such an arrangement allows the fixation element to be in a retracted position to minimize damage during delivery of the lead, and then moved to a deployed position, fixing the lead. After the distal end of the lead is fixed in place, the proximal end of the lead is connected to a port in the header of the CRM device.

While stylet-based delivery of cardiac leads is the most prevalent technique used, other techniques for cardiac lead delivery and fixation have also been developed. One such technique is an over-the-wire technique in which the lead is advanced over a guide wire. Different versions of this over-the-wire technique are described, for example, in U.S. Pat. Nos. 5,003,990, 5,304,218 and 6,129,749. Another technique involves the use of a guide catheter as a pusher for delivering the lead into position within the heart. Different versions of a guide catheter technique are described in U.S. Pat. Nos. 5,571,161, 6,185,464, 7,018,384 and 7,092,765.

In some situations, it may be necessary to explant a CRM device and the associated lead(s) from the patient. The device is explanted from the pectoral region and the lead is disconnected from the header of the device. Once disconnected from the CRM device, the lead presents a free end that can be conveniently accessed and utilized to extract the lead. In one approach, a cutting tool is introduced into the central lumen of the lead via the disconnected free end of the lead. In another approach, a cutting tool may be advanced over the free end of the lead body and advanced over or along the lead to a position proximate the lead tip. When positioned proximate the lead tip, the cutting device is used to sever the lead body from the tip, and the lead body may be extracted, leaving the tip implanted in the heart. Alternatively, the cutting tool may be used to cut away scar tissue from the area surrounding the tip. In a further approach, a catheter is introduced over the free end of the lead body and advanced toward the lead tip. The catheter is used to provide traction for pulling the lead from the heart.

Next generation long-term active implantable devices may take the form of elongated intravascular devices that are implanted within the patient's vascular system, instead of under the skin. Examples of these intravascular implantable devices (IID's) are described, for example, in U.S. Pat. No. 7,082,336 and U.S. Published Patent Application Nos. 2005/0043765A1, 2005/0228471A1 and 2006/0217779A1. These devices contain electric circuitry and/or electronic components that must be hermetically sealed to prevent damage to the electronic components and the release of contaminants into the bloodstream. Due to the length of these implantable devices, which in some cases can be approximately 10-60 cm in length, the devices must be flexible enough to move through the vasculature while being sufficiently rigid to protect the internal components.

In some embodiments, these intravascular implantable devices include cardiac leads that are coupled to one end of the elongated device body. The lead may be looped from the inferior end of the elongated device body residing in the vena cava, for example, up to the entrance into the right atrium, through the valve, and into the right ventricle. In these embodiments, the cardiac lead of an IID is unlike a cardiac lead for a conventional CRM device in that the proximal end of the lead is generally unavailable for access to aid in the implantation or explantation of the lead.

Because of these differences, lead introduction, fixation, and extraction devices and methods for conventional CRM devices are not necessarily applicable to intravascular implantable devices. For example, lead(s) for conventional CRM devices are usually introduced into the heart by way of the superior vena cava, while for intravascular implantable devices, the lead(s) are usually introduced to the heart via the inferior vena cava. The maneuvering of the lead from the inferior vena cava into the right atrium and on into the right ventricle is especially problematic using prior lead delivery systems and methods. Further, extraction techniques for conventional implantable CRM devices are unsuitable for use with intravascular implantable devices, as it may be difficult or impractical to access the lead body of an intravascular implantable device in order to sever the lead from its anchor, allowing extraction of the lead.

Previous approaches for delivering cardiac leads into the heart for intravascular implantable devices are disclosed in U.S. Pat. No. 7,082,336. In one approach, the lead includes a cuff, through which a guidewire is introduced through a distal end of the lead while the lead is outside of the body and the device is already implanted. The guidewire is steered to the fixation site, and a pusher is introduced onto the free end of the wire. The pusher is advanced against the lead cuff, and the lead is pushed along the guidewire to the fixation location. A fixation element is provided on the lead tip.

Further approaches for delivering cardiac leads into the heart for intravascular implantable devices are disclosed in application Ser. Nos. 12/327,791 and 12/327,808.A grasper-style tool is used to releasably grasp a distal end of the lead and guide the lead to the desired implant location. The lead is released from the grasper tool, and the tool is removed.

While the above approaches for implantation of leads for intravascular implantable devices are improvements over methods and devices for implanting leads for conventional CRM devices, a need still exists for further improved methods and devices for lead introduction, fixation, and extraction as they relate to intravascular implantable devices.

### SUMMARY OF THE INVENTION

In one embodiment, a lead delivery and fixation device is provided for use with an intravascular implantable device, including a sidecar having a selectively retractable fixation element. A lead is releasably coupled to the sidecar, with an electrode portion exposed for delivering a stimulation therapy. A manipulable catheter is coupled to the fixation element and configured to advance and withdraw a helix portion of the fixation element. The catheter and fixation element are offset from and generally parallel to the lead. The lead is separable from the sidecar in the event that extraction is required.

In one embodiment, a system for implanting a lead of an intravascular implantable device is provided, wherein the lead includes a proximal end attached to the intravascular implantable device and an electrode portion proximate a distal end. The system includes a steerable guide catheter having a torqueable driver therein and a catheter tip, and a sidecar apparatus having a first bore configured to receive the lead and a second bore including a bulkhead adapted to couple to the guide catheter tip, the second bore being substantially parallel to and axially offset from the first bore. The second bore includes of the sidecar apparatus having a selectively deployable fixation arrangement.

In one embodiment of a method of operating this system, the catheter is loaded into the sidecar, such that the driver is operably coupled to the fixation arrangement, which is in a retracted position. The lead is loaded into the sidecar, and the guide catheter is operated to deliver the sidecar, catheter, and lead to a desired implantation site. The fixation arrangement is moved from a retracted position to a deployed position. The driver is disconnected from the fixation arrangement, and the guide catheter including the driver is removed.

In one embodiment, the fixation arrangement is a fixation helix, and the driver is a stylet having a distal end that is adapted to interface with a proximal portion of the fixation helix to move the fixation helix from a retracted position to a deployed position. The lead may be releasably coupled to the sidecar, wherein a predetermined force is required to overcome the interface between the stylet and the proximal portion of the fixation helix to permit removal of the lead from the sidecar.

In one embodiment of a method of extracting the lead, a sheath or other tool is used to provide counter-traction for grasping the lead. The lead body may first be severed near its connection to the intravascular implantable device. A sheath may then be advanced over the lead body until the sheath abuts the sidecar. The sheath is used for counter-traction while the lead body is grasped with a tool and pulled from the sidecar, overcoming the o-ring connection of the lead in the sidecar. Alternatively, a tool may be advanced alongside the lead body, whether the lead is severed from the IID or not, and positioned against the sidecar. The tool is then used for counter-traction while the lead body is grasped with a tool and pulled from the sidecar, overcoming the o-ring connection of the lead in the sidecar.

In one embodiment, the present invention is an implantation system for an implantable intravascular medical device. The implantation system comprises an implantable intravascular medical device adapted for implantation within a vasculature of a patient, the implantable intravascular medical device including an elongated housing arrangement containing at least a power source and circuitry adapted to deliver medical therapy to the patient and a lead having a proximal portion operably connected to the elongated housing arrangement and a distal portion adapted to be positioned at a location within the patient. The implantation system further comprises a lead delivery system adapted for implanting the at least one lead within the patient, the lead delivery system including a catheter arrangement having a distal portion adapted for insertion into the vasculature of the patient and a proximal portion adapted to control the distal portion from a position external to the patient and a sidecar assembly, including a first longitudinal bore adapted to releasably receive the distal portion of the lead, a second longitudinal bore adapted to releasably receive the distal portion of the catheter arrangement and oriented generally parallel to the first longitudinal bore, and a fixation element disposed generally coaxially with the second longitudinal bore of the sidecar assembly and having structure extendable from the sidecar assembly adapted to facilitate securing the sidecar assembly at the location within the patient.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of one embodiment of the present invention.

FIG. 2 is a perspective sectional view of FIG. 1, depicting the active fixation arrangement in a deployed position.

FIG. 3 is a perspective view of another embodiment of the present invention featuring a passive fixation arrangement.

FIG. 4 is a perspective view of another embodiment of the present invention featuring a retracted fixation arrangement.

FIG. 5 is an exploded perspective view of an embodiment of the present invention.

FIG. 6 is an exploded perspective view of the sidecar, delivery catheter, and fixation arrangement according to an embodiment of the present invention.

FIG. 7 is a partial cutaway schematic view of one embodiment of the present invention during implantation.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

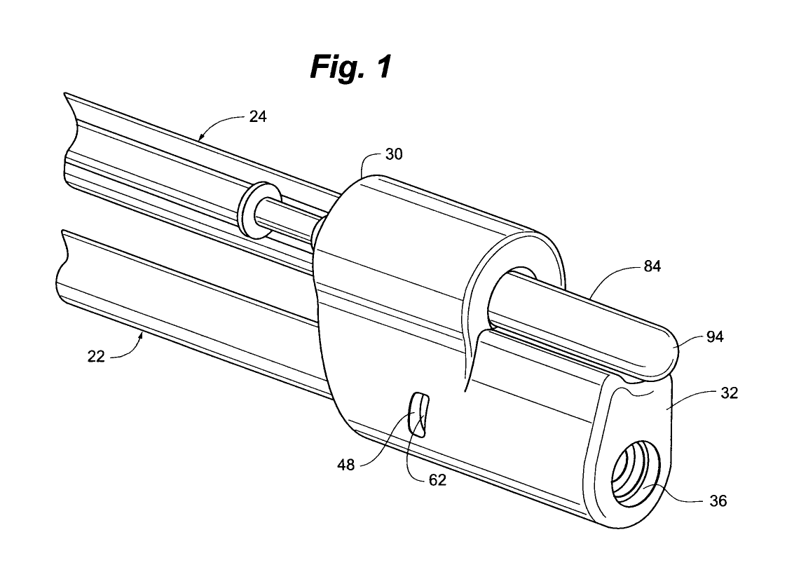

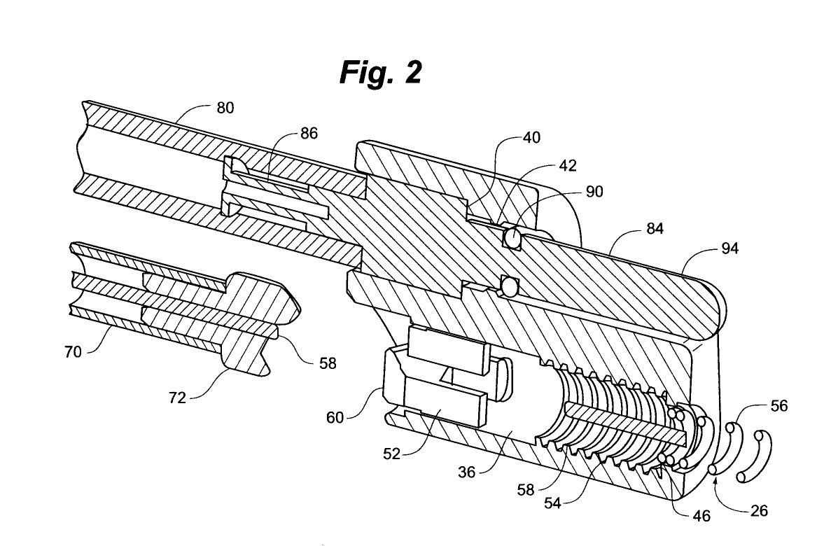

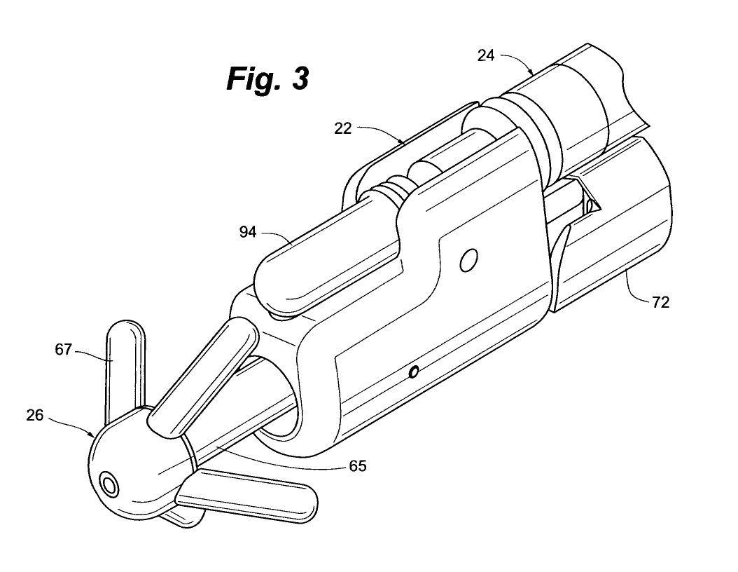

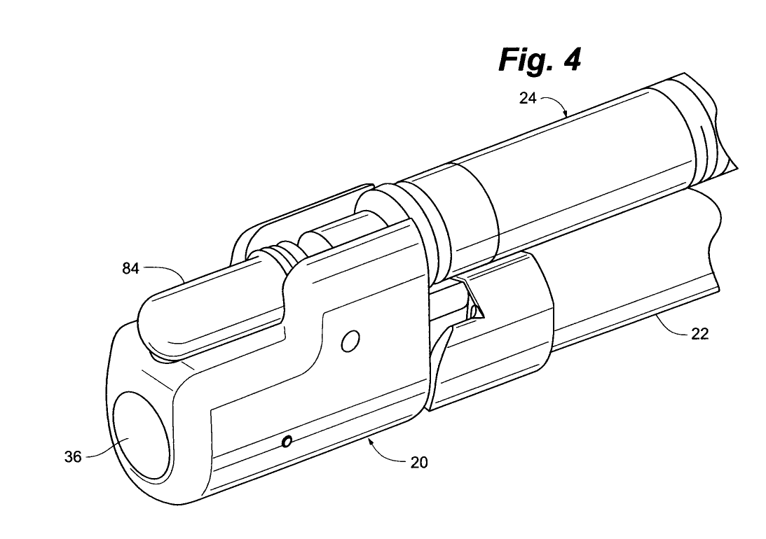

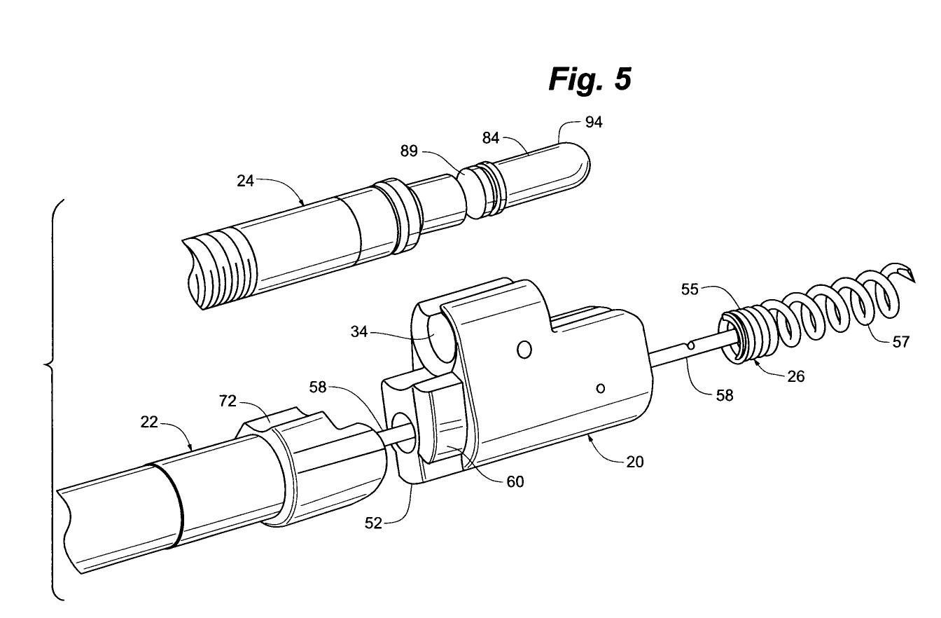

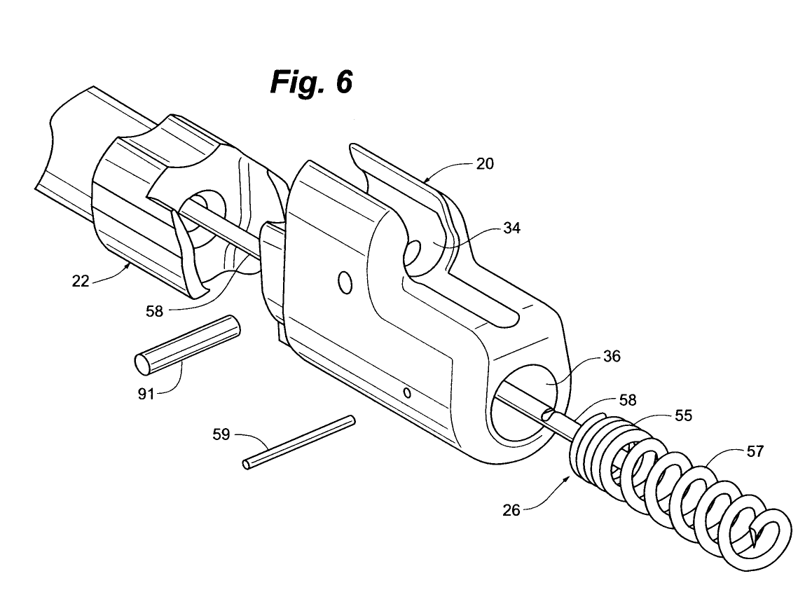

Referring to FIGS. 1-7, embodiments of the present invention is depicted, comprising a sidecar assembly 20, a delivery catheter 22, a lead 24, and a fixation arrangement 26. Sidecar assembly 20 is configured to facilitate the delivery, implantation, and extraction of lead 24 for use with an intravascular implantable device (IID) 28.

In one embodiment, the IID 28 includes components known in the art to be necessary to carry out the system functions. For example, the IID 28 may include one or more pulse generators, including associated batteries, capacitors, microprocessors, and circuitry for generating electrophysiological pulses for defibrillation, cardioversion and/or pacing. The IID 28 also includes detection circuitry for detecting arrhythmias or other abnormal activity of the heart. The specific components to be provided in the device will depend upon the application for the device, and specifically whether the device is intended to perform defibrillation, cardioversion and/or pacing along with its sensing functions.

The IID 28 comprises an elongated generally cylindrical housing proportioned to be passed into the vasculature and to be anchored within the patient's vasculature with minimal obstruction to blood flow. Suitable sites for the IID 28 may include, but are not limited to, the venous system using access through the right or left femoral vein or the subclavian or brachiocephalic veins, or the arterial system using access through one of the femoral arteries. Thus, the housing of IID 28 preferably has a streamlined maximum cross sectional diameter which may be in the range of 3-15 mm or less, with a most preferred maximum cross-sectional diameter of 3-8 mm or less. The cross-sectional area of the device 28 in the transverse direction (i.e. transecting the longitudinal axis) should be as small as possible while still accommodating the required components. The cross-section of the device 28 (transecting the longitudinal axis) may have a circular cross-section, although other cross-sections including crescent, flattened, or elliptical cross-sections may also be used. It can be desirable to provide the device with a smooth continuous contour so as to avoid voids or recesses that could encourage thrombus formation on the device.

Additional information pertaining to intravascular implantable devices can be found in U.S. Published Application Nos. 2005/0043765, 2008/0167702, and 2008/0147168, U.S. Pat. No. 7,082,336, and U.S. patent application Ser. No. 12/327,808, the disclosures of which are hereby incorporated by reference.

The proximal portion of lead 24 may be integrated with the IID device body, for example on the proximal end of the IID body, such that access to the end of lead 24 is generally unavailable. Lead 24 may also be included on the distal end of the device body, generally on the device body, and/or any combination thereof, as more than one lead 24 may be provided. In one embodiment, cardiac lead 24 generally includes one or more defibrillation and/or pacing electrodes and may also be equipped to sense electrical activity of the heart. Monitoring of the heart's electrical activity can be needed to detect the onset of an arrhythmia. Activity sensed by the sensing electrode(s) may be used by the device electronics to trigger delivery of a defibrillation shock. In this embodiment, cardiac lead 24 is functionally similar to a conventional defibrillation/pacing lead, although alternative lead configurations may be desirable if warranted by the desired placement of the IID 28 and cardiaclead 24 within the body.

The leads 24 may include non-thrombogenic and/or non-proliferative surfaces or coatings, for example, the leads 24 may include a coating that is anti-thrombogenic (e.g. perfluorocarbon coatings applied using supercritical carbon dioxide) so as to prevent thrombus formation on the lead 24. It is also beneficial for the coating to have anti-proliferative properties so as to minimize endothelialization or cellular ingrowth, since minimizing growth into or onto the lead 24 will help minimize vascular trauma when the device is explanted. The coating may thus also be one which elutes anti-thrombogenic compositions (e.g. heparin sulfate) and/or compositions that inhibit cellular in-growth and/or immunosuppressive agents.

Thus, it should be appreciated that in this disclosure the term “cardiac lead” is used to mean an element that includes conductors and electrodes and that thus may be positioned somewhat remotely from the circuitry that energizes the electrodes. In other embodiments, cardiac leads may include elements that are simply extensions or tapers of the IID itself (such as the portion of the device at which electrodes are located) as well as more conventional intravascular leads.

In another embodiment, the lead 24 may be provided with conduits, channels or passage ways for delivery of a fluid medicant, such as a drug or gene therapy, and IID 28 may be an implantable drug delivery device. In some embodiments, the lead 24 may include both a conduit or channel for fluids and also conductors for wires or the like for communication, sensing, control or delivery of electromagnetic stimulation.

In one embodiment, sidecar assembly 20 generally comprises a proximate portion 30, a distal portion 32, a first longitudinal bore 34 and a second longitudinal bore 36. First bore 34 is configured to retain lead 24. In one embodiment, the distal end of lead 24 is non-releasably retained within first bore 34 such that lead 24 and sidecar 20 are inseparable. This may be accomplished in manufacturing, such as by welding or molding, or may be accomplished after manufacture in an assembly step. Sidecar 20 may be electrically insulative. Although it is contemplated that sidecar 20 and/or fixation arrangement 26 could be configured to be electrically conductive, electrical stimulation into scar tissue is generally less effective than stimulation of healthy tissue. Therefore, the arrangement of sidecar 20 with lead 24 and electrode portion 94 axially offset from fixation arrangement 26 provides the ability for stimulation delivered through lead 24 and electrode portion 94 to more effectively capture healthy cardiac tissue instead of scar tissue.

In another embodiment, first bore 34 is configured to releasably retain the distal end of lead 24, such that lead 24 is securely joined to sidecar 20 while implanted, but is capable of being separated from sidecar 20 by pulling lead 24 with a predetermined amount of force. In such an embodiment, suitable arrangements for the releasable connection between lead 24 and first bore 34 may include components such as a ball detent, interference fit, an O-ring, and/or a snap ring.

Second longitudinal bore 36 of sidecar 20 is configured to retain delivery catheter 22 and/or fixation arrangement 26, and second bore 36 is generally parallel to and axially offset from first bore 34. Fixation arrangement 26 may comprise a passive fixation element or an active fixation element, and fixation arrangement 26 may be selectively deployable from second bore 36, or may be provided in an unmovable deployed configuration. Referring to passive fixation embodiments, fixation arrangement 26 may comprise a conventional tined tip, as is known in the art of passive fixation leads. Tined tip is retained within and protrudes distally out of second bore 36, and is not movable into or out of second bore 36. In another embodiment, passive fixation arrangement 26 is selectively deployable from second bore 36 and generally includes a shaft 65 with a plurality of automatically extending tines 67. Suitable tines 67 may be spring-loaded, or made from shape memory alloy, or made from pliable material such as silicone, such that when fixation arrangement 26 is in a retracted position within second bore 36, tines 67 are generally folded up against fixation shaft 65. As fixation arrangement 26 is moved from a retracted position within second bore 36 to a deployed position, tines 67 will fold out from fixation shaft 65.

Referring now to an active fixation embodiment, fixation arrangement 26 may be selectively deployable from second bore 36, or may be provided in an unmovable, deployed position. A selectively deployable configuration has the advantage of allowing fixation arrangement 26 to be retracted within sidecar 20 during delivery of lead 24 and sidecar 20, preventing possible damage to tissue during delivery. Once at the desired location, fixation arrangement 26 is moved from the retracted position to a deployed position. In another embodiment, fixation arrangement 26 includes a helix 56 non-retractably coupled to second bore 36, such that helix 56 protrudes distally from sidecar 20. Helix 56 may be coated with a bio-soluble compound that covers the sharp end of helix 56, preventing damage to the vasculature during delivery of lead 24 and sidecar 20. Fixation arrangement 26 may also include a rotatable slipjoint, to allow rotation of helix 56 about a longitudinal axis to facilitate introduction of helix 56 into tissue at a desired implant location.

Referring now to selectively deployable active fixation arrangements, in one embodiment fixation arrangement 26 comprises a bulkhead 52, threaded screw portion 54, helix 56, and driver 58. Bulkhead 52 includes an interface portion 60 configured to interact with catheter 22, a plurality of tabs 62 configured to retain bulkhead 52 in sidecar 20, and a central passage through which driver 58 may pass. Additional retention features may be provided on bulkhead 52 to prevent unwanted rotation, translation, or other movement of bulkhead 52 in sidecar 20. In one embodiment, the distal end of driver 58 is fixed to screw portion 54 during manufacturing, such as by welding or crimping. Helix 56 is also fixed to screw portion 54, such as by welding, or helix 56 may be integrated with screw 54. Bulkhead 52 also functions to prevent fixation arrangement 26 from backing out of the proximal portion 30 of sidecar 20. In such an embodiment, sidecar 20 includes a second bore 36 having a threaded portion 44 to receive screw 54, a shoulder 46 to provide a distal stop for screw 54, and one or more slots 48 to receive tabs 62.

In another embodiment, a helix 57 is provided having a threaded portion 55, and sidecar 20 includes a transverse pin 59 configured to act as a stop, preventing over-deployment of helix 57.

Catheter 22 may be configured to releasably couple to second bore 36 at the proximal end of sidecar 20, and comprises a body portion 70 having a driver 58 and internal pull wires to provide articulation and/or extension of catheter 22, and a distal tip 72. The proximal end of catheter 22 is coupled to a control handle maintained outside of the patient during a procedure, the control handle including means for activating the articulation, extension, and rotation of catheter 22 as well as activation of driver 58. Catheter tip 72 is configured to selectively couple to interface portion 60 of bulkhead 52, securing catheter 22 during delivery of sidecar 20 and lead 24, and during manipulation of fixation arrangement 26.

In one embodiment, driver 58 is maintained mostly within catheter 22, with the distal end of driver 58 being coupled to fixation arrangement 26, such as by passing through bulkhead 52 and being fixedly secured to screw 54. In such an embodiment, driver 58 includes a break feature such as a notch, so that after successful deployment of fixation arrangement 26 with driver 58 such that screw 54 is engaged against stop 46, driver 58 is overtorqued causing driver to break at the notch and allowing removal of catheter 22 from second bore 36.

In another embodiment, driver 58 is selectively engageable with fixation arrangement 26. For example, the distal end of driver 58 may be provided with a shape profile suitable for transmitting torque, such as a flathead screwdriver profile, or hex profile, or square profile, or other like configuration. In another embodiment, driver 58 may comprise a removable stylet tool, configured to be introduced into catheter 22 and engaged with fixation arrangement 26 for deployment of the fixation arrangement, and then removed from catheter 22.

As mentioned above, lead 24 may be releasably coupled to sidecar 20 in first bore 34. In one embodiment, lead 24 includes a body portion 80 having a conductor within a passage 82, and a tip portion 84 coupled to the distal end of body 80. Tip 84 may include a crimp area 86, and a flared portion, such that lead 24 may be crimped onto tip portion 84. Tip 84 further includes a robust profile 88, configured to seat against a shoulder 40 in first bore 34 of sidecar 20. An O-ring 90 is provided on tip 84, which combines with a throat portion 42 of sidecar 20 to provide an interference fit, preventing the accidental pull-out of lead 24 from sidecar 20. The relative sizes, material composition, and material hardness of O-ring 90 and throat 42 are among the characteristics that can be selected to determine a minimum required force necessary to remove lead 24 from sidecar 20. In other embodiments, O-ring 90 may be replaced with a snap-ring or other arrangement to facilitate releasable retention of tip 84 in sidecar 20.

In another embodiment, tip 84 includes a circumferential groove 89 and first bore 34 includes a spring-loaded ball (not shown) to create a ball detent connection between lead 24 and sidecar 20. In a further embodiment, a pin 91 is provided transversely across a portion of first bore 34, to interact with groove 89 on tip 84. To assemble lead 24 into first bore 34, pin 91 may be removable such that lead tip 84 is advanced into first bore 34, and pin 91 is inserted to retain lead 24. Alternatively, pin 91 may remain installed, and lead 24 is simply snapped into place. First bore 34 may optionally include a longitudinal channel 93, to allow temporary enlargement of first bore 34 upon advancement of lead 24 over pin 91.

Lead tip 84 further includes a conductor input and an electrode portion 94. Although tip 84 is constructed entirely of electrically conductive material, the exposed portion proximate the distal end of tip 84 will be referred to as electrode 94. Electrode portion 94 may be configured as a unipolar electrode or may be provided with an additional band electrode spaced apart from the tip to be configured as a bipolar electrical arrangement.

The exposed portion of electrode 94 increases the surface area from which therapy is delivered. Additionally, electrode 94 may include surface modification techniques to increase its surface area, and/or include a well for drugs such as steroids. As depicted in the Figures, electrode portion 94 may extend beyond the distal end of sidecar 20. Conductor input is configured to receive conductor (not shown), electrically coupling electrode 94 to a pulse generator (not shown).

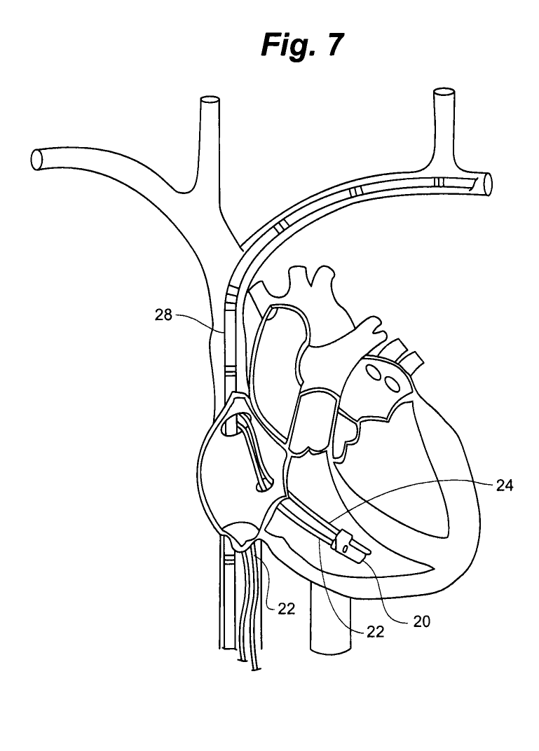

Referring now to the implantation of the various embodiments of the present invention, intravascular implantable device 28 may be implanted prior to, or subsequent to, implantation and fixation of sidecar 20 and lead 24. In one embodiment, IID 28 is first implanted, and is provided with an integrated lead 24 on its proximal, or inferior, end. Delivery catheter 22 and lead 24 are coupled to sidecar 20, and fixation arrangement 26 is in a retracted position within sidecar 20. Catheter 22 is manipulated to guide sidecar 20 and lead 24 into the desired location. In one embodiment lead 24 is introduced from the inferior vena cava, into the right atrium, and on to the right ventricle, as depicted in FIG. 7. In another embodiment, lead 24 is introduced from the superior vena cava. In a further embodiment, lead 24 may be guided to the coronary sinus.

When sidecar 20 and lead 24 are guided to the desired location within the patient, the surgeon is able to test the electrical performance of lead 24 prior to deployment of the helix fixation element if desired, due to electrode 94 protruding beyond sidecar 20. Once the performance of lead 24 is satisfactory, helix 56 is deployed by manipulating the torque means in catheter 22, causing driver 58 to advance screw portion 54 through threaded portion 44 until screw 54 bottoms out against shoulder 46. If desired, additional electrical performance testing may be undertaken at this time, as helix 56 is still capable of being retracted, allowing repositioning of lead 24 and sidecar 20.

When it is desired to remove catheter 22, in the embodiment driver 58 is provided with a break feature such as a notch on the shaft, driver 58 is overtorqued causing it to break at the notch. In other embodiments discussed herein, driver 58 is simply retracted from screw 54. Catheter 22 can then be withdrawn, leaving lead 24 and sidecar 20 implanted in the patient.

In some circumstances, it may be necessary to extract lead 24 from a patient. In an embodiment wherein lead 24 is releasably coupled to sidecar 20, lead 24 may be extracted while sidecar 20 and fixation arrangement 26 are left within the patient. The miniature size of sidecar 20, and the nature of the bio-compatible materials renders sidecar 20 and fixation arrangement 26 safe for long-term retention in the patient. To detach lead 24, it may be possible to simply pull on lead body 80 with a tool to dislodge tip portion 84 from sidecar 20. The use of a sheath or other mechanism may be required for counter-traction to defeat the connection between lead 24 and sidecar 20.

In an embodiment wherein lead 24 is non-releasably coupled to sidecar 20, extraction of lead 24 is accomplished by introduction of a cutting tool to cut the distal end of lead 24. Sidecar 20 and fixation arrangement 26 remain in the patient, while severed lead 24 is removed. Similarly, if it is determined that a lead 24 releasably coupled to sidecar 20 cannot be separated from sidecar 20 by pulling, a cutting tool may be used to sever lead 24, allowing extraction.

In one embodiment, sidecar 20 may be provided with a drug reservoir or drug-eluting structure for the release of medicaments into a patient. Such an embodiment may be used in a cardiac implantation and include anti-inflammatory or anti-thrombogenic agents. Alternatively, the embodiment may be implanted at a different location within the patient, such as in or proximate the kidneys, for the delivery of therapeutic drugs either as a standalone therapy, or in combination with an electrical therapy. Additional information pertaining to drug delivery and drug reservoirs for intravascular implantable devices can be found in U.S. Published Patent Application No. 2007/0255379, the disclosure of which is hereby incorporated by reference.

In one embodiment, instructions for implanting the lead 24 in accordance with the various embodiments described herein in the form of printed or electronically, optically or magnetically stored information to be displayed, for example, are provided as part of a kit or assemblage of items prior to surgical implantation of the lead 24. In another embodiment, instructions for implanting the lead 24 in accordance with the various embodiments described herein are provided, for example, by a manufacturer or supplier of lead 24, separately from providing the lead 24, such as by way of information that is accessible using the Internet or by way of seminars, lectures, training sessions or the like.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. An implantation system for an implantable intravascular medical device, comprising:
an implantable intravascular medical device adapted for implantation within a vasculature of a patient, the implantable intravascular medical device including:
an elongated housing arrangement containing at least a power source and circuitry adapted to deliver medical therapy to the patient; and
a lead having a proximal portion operably connected to the elongated housing arrangement and a distal portion adapted to be positioned at a location within the patient; and
a lead delivery system adapted for implanting the at least one lead within the patient, the lead delivery system including:
a catheter arrangement having a distal portion adapted for insertion into the vasculature of the patient and a proximal portion adapted to control the distal portion from a position external to the patient; and
a sidecar assembly, including a first longitudinal bore adapted to releasably receive the distal portion of the lead, a second longitudinal bore adapted to releasably receive the distal portion of the catheter arrangement and oriented generally parallel to the first longitudinal bore, and a fixation element disposed generally coaxially with the second longitudinal bore of the sidecar assembly and having structure extendable from the sidecar assembly adapted to facilitate securing the sidecar assembly at the location within the patient.

2. The implantation system of claim 1, wherein the fixation element includes an active fixation member that is selectively extendable from a retracted position to a deployed position by operation of the catheter arrangement.

3. The implantation system of claim 2, wherein the fixation element is a helix member and the catheter arrangement further includes a stylet insertable through a lumen in the catheter to operably engage the helix member and move the helix member from the retracted position to the deployed position.

4. The implantation system of claim 1, wherein the distal portion of the lead includes an electrode and the sidecar assembly is configured to retain the distal portion of the lead such that the electrode is at least partially exposed to the patient.

5. The implantation system of claim 1, wherein sidecar assembly is configured such that a retraction force applied to the lead in a direction from the distal portion to the proximal portion will releasably disengage the distal portion of the lead from the sidecar only when the retraction force is greater than a fixation force that retains the fixation element at the location, the fixation force being determined after a period of implantation of the implantable medical device of at least a week.

6. An implantation system for an implantable intravascular medical device, comprising:
an implantable intravascular medical device adapted for implantation within a vasculature of a patient, the implantable intravascular medical device including:
an elongated housing arrangement containing at least a power source and circuitry adapted to deliver medical therapy to the patient; and
a lead having a proximal portion operably connected to the elongated housing arrangement and a distal portion adapted to be positioned at a location within the patient; and
a lead delivery system adapted for implanting the at least one lead within the patient, the lead delivery system including:
a delivery means having a distal portion adapted for insertion into the vasculature of the patient and a proximal portion adapted to control the distal portion from a position external to the patient; and
a carrier means, including a lead retention means adapted to releasably receive the distal portion of the lead, a delivery retention means adapted to releasably receive the distal portion of the delivery means and oriented generally parallel to the lead retention means, and a fixation means offset from the lead retention means and adapted to facilitate securing the carrier means with the lead at the location within the patient.

7. A method of implanting a lead associated with an intravascular implantable device having an elongated housing arrangement containing at least a power source and circuitry adapted to deliver medical therapy to the patient, wherein the lead includes a proximal potion operably connected to the elongated housing arrangement, the method comprising:
coupling a distal portion of a delivery catheter to a sidecar assembly, wherein a distal portion of the lead is retained within a first longitudinal bore of the sidecar assembly and wherein the sidecar assembly includes a second longitudinal bore adapted to releasably receive the distal portion of the delivery catheter and oriented generally parallel to the first longitudinal bore, and a fixation element disposed generally coaxially with the second longitudinal bore of the sidecar assembly;
manipulating the delivery catheter to advance the sidecar assembly and lead through a vasculature of the patient to a desired location;
using the delivery catheter to deploy the fixation element to secure the sidecar assembly at the desired location within the patient; and
removing the distal portion of the delivery catheter from the sidecar assembly and withdrawing the delivery catheter from the patient.

8. A method of implanting a lead associated with an intravascular implantable device, comprising:
providing an implantable intravascular medical device adapted for implantation within a vasculature of a patient, the implantable intravascular medical device including:
an elongated housing arrangement containing at least a power source and circuitry adapted to deliver medical therapy to the patient; and
a lead having a proximal portion operably connected to the elongated housing arrangement and a distal portion adapted to be positioned at a location within the patient;
providing a lead delivery system adapted for implanting the at least one lead within the patient, the lead delivery system including:
a catheter arrangement having a distal portion adapted for insertion into the vasculature of the patient and a proximal portion adapted to control the distal portion from a position external to the patient; and
a sidecar assembly, including a first longitudinal bore adapted to retain the distal portion of the lead, a second longitudinal bore adapted to releasably receive the distal portion of the catheter arrangement and oriented generally parallel to the first longitudinal bore, and a fixation element disposed generally coaxially with the second longitudinal bore of the sidecar assembly; and
providing instructions for implanting the lead, including:
coupling the distal portion of the catheter arrangement to the second longitudinal bore;
advancing the lead and the sidecar assembly to a desired location with the catheter arrangement;
deploying the fixation element at the desired location; and
removing the distal portion of the delivery catheter from the sidecar assembly and withdrawing the delivery catheter from the patient.

9. A method of extracting a lead from within a patient, as substantially shown and described herein.
